(19) United States
(12) Patent Application Publication
(10) Pub. No.: US 2021/0278403 A1
(43) Pub. Date: Sep. 9, 2021

(54) LATERAL FLOW ASSAY FOR ASSESSING RECOMBINANT PROTEIN EXPRESSION OR REPORTER GENE EXPRESSION

(71) Applicant: Qoolabs, Inc., San Diego, CA (US)

(72) Inventor: Hong Qi, San Diego, CA (US)

(73) Assignee: Qoolabs, Inc., San Diego, CA (US)

(21) Appl. No.: 16/326,369

(22) PCT Filed: Aug. 17, 2017
(86) PCT No.: PCT/US2017/047445

§ 371 Date: Feb. 18, 2019

(51) Int. Cl. G01N 33/558; G01N 33/53

(52) U.S. Cl. CPC G01N 33/558; G01N 33/5302

(57) ABSTRACT

The present disclosure relates to lateral flow test devices, and uses thereof, for assessing recombinant protein (polypeptide) expression or reporter gene expression in a sample. In some aspects, the present disclosure relates to the use of lateral flow immunoassay to quickly detect the expression of recombinant proteins. In some aspects, lateral flow immunoassay can be used for qualitative and/or quantitative analysis of the recombinant protein expression.

### CROSS REFERENCE TO RELATED APPLICATION

This application claims priority to U.S. Provisional Application No. 62/378,538, filed 23 Aug. 2016, having the title “Lateral Flow Assay for Assessing Recombinant Protein Expression or Reporter Gene Expression,” the content of which application is incorporated herein by reference in its entirety for all purposes.

### FIELD

The present disclosure relates to lateral flow test devices, and uses thereof, for assessing recombinant protein (polypeptide) expression or reporter gene expression in a sample. In some aspects, the present disclosure relates to the use of lateral flow immunoassay to quickly detect the expression of recombinant proteins. In some aspects, lateral flow immunoassay can be used for qualitative and/or quantitative analysis of the recombinant protein expression.

### BACKGROUND

Recombinant protein expression is a widely used method for protein production. The ability to produce large amount of recombinant proteins makes possible to study proteins that are not naturally abundant. Recombinant protein expression is also widely used in biotechnology and pharmaceutical industry in the scaled-up production of proteins of interest. It is often necessary to examine the expression level of intended protein quickly during expression and purification. Currently the most commonly used methods to achieve this goal include SDS-PAGE, western blot analysis, ELISA etc. The time needed to complete the testing often takes several hours or longer. A system or device that could determine recombinant protein expression level in minutes will be a highly valuable tool during recombinant protein expression.

Lateral flow immunoassay (LFIA) offer several advantages over other detection methods. Lateral flow immunoassay devices detect antigen or antibody in samples within minutes. It is relatively inexpensive and maintains long-term stability over a wide range of climates. It is simple to use and requires little or no samples/reagents preparations. No additional handling equipment is required.

LFIA has been used in point-of-care or point-of-use diagnosis and detection for over twenty years. The most well-known LFIA device is the in-home pregnancy test. LFIA has been widely used in rapid test diagnosis, food contaminant detection, and drug abuse screenings. For example, U.S. Pat. No. 5,714,341 A, filed on Mar. 30, 1994, discloses the use of LFIA for HIV specific antibodies in saliva samples; U.S. patent application publication No. US 2004/0184954 A1, filed on Nov. 19, 2003, discloses the use of LFIA for drug abuse test from saliva samples; and U.S. Pat. No. 7,749,772 B1, priority dated Jun. 29, 2006, discloses the use of LFIA for the detection of Δ9-Tetrahydrocannabinol in body fluids (including saliva) for drug abuse screening. Other patents relating to LFIA include U.S. Pat. Nos. 5,073,464, 5,073,484 and 5,559,041.

There is a need for improved assays for assessing recombinant protein (polypeptide) expression or reporter gene expression in a sample, e.g., assays that are less time-consuming. The present disclosure addresses this and the related needs.

### SUMMARY

In one aspect, the present disclosure provides a lateral flow test device for assessing recombinant protein (polypeptide) expression or reporter gene expression in a sample, which device comprises a porous matrix that comprises a test location on said porous matrix, said test location comprising a test reagent that binds to an analyte or another binding reagent that binds to said analyte, or is an analyte or an analyte analog that competes with an analyte in said sample for binding to a binding reagent for said analyte, wherein said analyte is a recombinant protein (polypeptide) expression product or a reporter gene expression product, and wherein a liquid sample flows laterally along said test device and passes said test location to form a detectable signal to determine the presence, absence and/or amount of said recombinant protein (polypeptide) expression product or said reporter gene expression product in said sample.

In some embodiments, the present disclosure relates to the use of lateral flow immunoassay (LFIA) device to quickly detect the expression of recombinant proteins. In a preferred embodiment, the LFIA device (test strip) comprises or consists of four elements (sample pad, conjugate pad, nitrocellulose membrane, absorbent pad) mounted consecutively on a solid backing. Colloidal gold nanoparticles conjugated to antibodies specific to protein or epitope tags are deposited onto the conjugate pad, which forms an immuno-complex with recombinant proteins expressing protein or epitope tags in the sample. A second antibody specific to protein or epitope tags is immobilized onto the nitrocellulose membrane. The second antibody captures the first immuno-complex. Accumulation of chromatic colloidal gold nanoparticles of the immuno-complex allows detection of recombinant protein expression.

Any suitable matrix can be used in the present devices. For example, the matrix can comprise nitrocellulose, glass fiber, polypropylene, polyethylene (preferably of very high molecular weight), polyvinylidene flouride, ethylene vinylacetate, acrylonitrile and/or polytetrafluoro-ethylene.

Any suitable test reagent can be used in the present devices. For example, the test reagent can bind to the analyte. In some embodiments, the test reagent specifically binds to the analyte. In another example, the test reagent can bind to another binding reagent that binds to the analyte. In still another example, the test reagent can be an analyte or an analyte analog that competes with an analyte in the sample for binding to a binding reagent for the analyte.

The test reagent can be any suitable substance. For example, the test reagent can be a peptide or a protein. In some embodiments, the protein can be an antigen or an antibody, e.g., an antibody that specifically binds to a recombinant protein (polypeptide) expression product or a reporter gene expression product.

The matrix can be in any suitable form. For example, the matrix can be in the form a strip or a circle. The matrix can be a single element or can comprise multiple elements.

The present test device can further comprise a sample application element upstream from and in fluid communication with the matrix. The present test device can further comprise a liquid absorption element downstream from and in fluid communication with the matrix. The present test device can further comprise a control location.

At least a portion of the matrix can be supported by a solid backing. In some embodiments, the entire matrix can be supported by a solid backing.

A portion of the matrix, upstream from the test location, can comprise a dried, labeled reagent, the labeled reagent being capable of being moved by a liquid sample and/or a further liquid to the test location and/or the control location to generate a detectable signal. The dried, labeled reagent can be located at any suitable location. For example, the dried, labeled reagent can be located downstream from a sample application place on the test device. In another example, the dried, labeled reagent can be located upstream from a sample application place on the test device.

The present test device can further comprise, upstream from the test location, a conjugate element that comprises a dried, labeled reagent, the labeled reagent being capable of moved by a liquid sample and/or a further liquid to the test location and/or the control location to generate a detectable signal. The conjugate element, e.g., a conjugate pad, can be located at any suitable location. For example, the conjugate element can be located downstream from a sample application place on the test device. In another example, the conjugate element can be located upstream from a sample application place on the test device.

The present test device can comprise any suitable labeled reagent. For example, the labeled reagent can bind, and preferably specifically binds, to an analyte in the sample.

Any suitable label can be used. For example, the label can be a soluble label, e.g., such as a colorimetric, radioactive, enzymatic, luminescent or fluorescent label. In another example, the label can be a particle or particulate label, such as a particulate direct label, or a colored particle label. Exemplary particle or particulate labels include colloidal gold label, latex particle label, nanoparticle label and quantum dot label.

The labeled reagent can be dried in the presence of a material that: a) stabilizes the labeled reagent; b) facilitates solubilization or resuspension of the labeled reagent in a liquid; and/or c) facilitates mobility of the labeled reagent. Any suitable material can be used. For example, the material can be a protein, e.g., a casein or BSA, a peptide, a polysaccharide, a sugar, a polymer, e.g., polyvinylpyrrolidone (PVP-40), a gelatin or a detergent, e.g., Tween-20.

The analyte and/or the labeled reagent can be transported to the test location by any suitable means. For example, a sample liquid alone can be used to transport the analyte and/or the labeled reagent to the test location. In another example, a developing liquid can be used to transport the analyte and/or the labeled reagent to the test location.

The present test device can further comprise a housing that covers at least a portion of the test device, wherein the housing comprises a sample application port to allow sample application upstream from or to the test location and an optic opening around the test location to allow signal detection at the test location. In some embodiments, the housing covers the entire test device. In other embodiments, at least a portion of the sample receiving portion of the matrix or the sample application element is not covered by the housing and a sample is applied to the portion of the sample receiving portion of the matrix or the sample application element outside the housing and is then transported to the test location.

The housing can comprise any suitable material. For example, the housing can comprise a plastic material.

The present test device can be configured for a sandwich assay. In some embodiments, the test reagent and the labeled reagent bind to the analyte, and at least one of the test reagent and the labeled reagent specifically binds to the analyte. In some examples, the test reagent and the labeled reagent specifically bind to the analyte.

The present test device can also be configured for a competition assay. In some embodiments, the test reagent is an analyte or an analyte analog that competes with an analyte in the sample for binding to the labeled reagent that binds to the analyte. In some examples, the labeled reagent specifically binds to the analyte. In other embodiments, the test reagent binds to the analyte and the labeled reagent comprises an analyte or an analyte analog that competes with an analyte in the sample for binding to the test reagent. In some examples, the test reagent specifically binds to the analyte.

The present test device can be configured for assessing recombinant protein expression in a sample. The present test device can be configured as a sandwich assay for assessing recombinant protein expression in a sample. In some embodiments, the test reagent and the labeled reagent bind to the recombinant protein expression product, and at least one of the test reagent and the labeled reagent specifically binds to the recombinant protein expression product. The recombinant protein expression product to be assessed can comprise a target protein portion and a tag portion. In some examples, at least one of the test reagent and the labeled reagent binds to, or specifically binds to, the target protein portion. In other examples, at least one of the test reagent and the labeled reagent binds to, or specifically binds to, the tag portion.

The present test device can also be configured as a competition assay for assessing recombinant protein expression in a sample. In some embodiments, the test reagent is the recombinant protein expression product, or a portion thereof, that competes with the recombinant protein expression product in the sample for binding to the labeled reagent that binds to, or specifically binds to, the recombinant protein expression product. The recombinant protein expression product to be assessed can comprise a target protein portion and a tag portion. In some examples, the test reagent comprises the target protein portion of the recombinant protein expression product, or a portion thereof, and the labeled reagent binds to, or specifically binds to, the target protein portion the recombinant protein expression product. In other examples, the test reagent comprises the tag portion of the recombinant protein expression product, or a portion thereof, and the labeled reagent binds to, or specifically binds to, the tag portion.

The present test device can be configured for assessing reporter gene expression in a sample. The present test device can be configured as a sandwich assay for assessing reporter gene expression in a sample. In some embodiments, the test reagent and the labeled reagent bind to the reporter gene expression product, and at least one of the test reagent and the labeled reagent specifically binds to the reporter gene expression product. The reporter gene expression product can comprise a reporter protein portion and a tag portion. In some examples, at least one of the test reagent and the labeled reagent binds to, or specifically binds to, the reporter protein portion. In other examples, at least one of the test reagent and the labeled reagent binds to, or specifically binds to, the tag portion.

The present test device can also be configured as a competition assay for assessing reporter gene expression in a sample. In some embodiments, the test reagent is the reporter gene expression product, or a portion thereof, that competes with the reporter gene expression product in the sample for binding to the labeled reagent that binds to, or specifically binds to, the reporter gene expression product. The reporter gene expression product can comprise a reporter protein portion and a tag portion. In some examples, the test reagent comprises the reporter protein portion of the reporter gene expression product, or a portion thereof, and the labeled reagent binds to, or specifically binds to, the reporter protein portion of the reporter gene expression product. In other examples, the test reagent comprises the tag portion of the reporter gene expression product, or a portion thereof, and the labeled reagent binds to, or specifically binds to, the tag portion of the reporter gene expression product.

The present test device can be configured for any suitable uses or applications. For example, the present test device can be configured for: 1) assessing multiple recombinant protein expression products or multiple reporter gene expression products in a sample; 2) determining a candidate for recombinant protein expression with intended production yield; 3) assessing recombinant protein expression or reporter gene expression time course; 4) optimizing induction condition for recombinant protein expression or reporter gene expression; and/or 5) determining the fraction(s) with intended recombinant protein during protein purification, e.g., chromatography.

In another aspect, the present disclosure provides a method for assessing recombinant protein expression or reporter gene expression in a sample, which method comprises: a) contacting a liquid sample with a test device described above, wherein the liquid sample is applied to a site of the test device upstream of the test location; b) transporting the analyte, if present in the liquid sample, and a labeled reagent to the test location; and c) assessing a detectable signal at the test location to determine the presence, absence and/or amount of said recombinant protein expression product or said reporter gene expression product in said sample.

The present methods can be used in any suitable format. In some embodiments, the liquid sample and the labeled reagent can be premixed to form a mixture and the mixture is applied to the test device. The present methods can further comprise a washing step after the mixture is applied to the test device. The washing step can be conducted in any suitable manner. For example, the washing step can comprise adding a washing liquid after the mixture is applied to the test device. In another example, the test device can comprise a liquid container comprising a washing liquid and the washing step comprises releasing the washing liquid from the liquid container.

In some embodiments, the present test device can comprise a dried labeled reagent before use and the dried labeled reagent can be solubilized or resuspended, and transported to the test location by the liquid sample. The dried labeled reagent can be located at any suitable location. For example, the dried labeled reagent can be located downstream from the sample application site, and the dried labeled reagent can be solubilized or resuspended, and transported to the test location by the liquid sample. In another example, the dried labeled reagent can be located upstream from the sample application site, and the dried labeled reagent can be solubilized or resuspended, and transported to the test location by another liquid.

The analyte and/or labeled reagent can be solubilized or resuspended, and transported to the test location by any suitable means. For example, the labeled reagent can be solubilized or resuspended, and transported to the test location by the liquid sample alone. In another example, the analyte and/or labeled reagent can be solubilized or resuspended, and transported to the test location by another liquid.

The present methods can be used for assessing recombinant protein expression or reporter gene expression in any suitable sample. For example, the present methods can be used for assessing recombinant protein expression or reporter gene expression in a liquid sample that comprises a cell lysate, a cell culture medium, an in vitro transcription product, an in vitro translation product, a polypeptide purification fraction, and/or a sample isolated or derived from a subject. In some embodiments, the present methods can be used for assessing recombinant protein expression product or reporter gene expression product obtained from in vitro transcription, in vitro translation, cell(s) or cell line(s). In other embodiments, the present methods can be used for assessing recombinant protein expression product or reporter gene expression product obtained from in vivo expression, e.g., recombinant protein expression product or reporter gene expression product in a subject. The exemplary subject can be a human, or a non-human subject such as an experimental animal, a pet or a farm animal. For example, the present methods can be used for assessing recombinant protein expression product or reporter gene expression product in a sample isolated or derived from a subject. The present methods can be used for any suitable purposes, e.g., monitoring drug metabolism in a subject or a patient, or as part of a drug screening or discovery process.

The detectable signal can be assessed by any suitable means. For example, the detectable signal can be assessed by visual inspection by a user. In another example, the detectable signal can be assessed by a reader. Any suitable reader can be used. In some embodiments, the detectable signal is a fluorescent signal and the fluorescent signal is assessed by a fluorescent reader. Any suitable fluorescent reader can be used. For example, the fluorescent reader can be a laser based or a light emitting diode (LED) based fluorescent reader. The reader comprises a single or multiple photodetectors.

The present method can be conducted for any suitable uses or applications. In some embodiments, the present method can be conducted for assessing a recombinant protein expression in a sample. For example, the present method can be conducted for determining the presence or absence of the recombinant protein expression product in the sample. In another example, the present method can be conducted for determining the amount of the recombinant protein expression product in the sample. In other embodiments, the present method can be conducted for assessing a reporter gene expression in a sample. For example, the present method can be conducted for determining the presence or absence of the reporter gene expression product in the sample. In another example, the present method can be conducted for determining the amount of the reporter gene expression product in the sample. In still other embodiments, the present method can be conducted for: 1) assessing multiple recombinant protein expression products or multiple reporter gene expression products in a sample; 2) determining a candidate for recombinant protein expression with intended production yield; 3) assessing recombinant protein expression or reporter gene expression time course; 4) optimizing induction condition for recombinant protein expression or reporter gene expression; and/or 5) determining the fraction(s) with intended recombinant protein during protein purification, e.g., chromatography.

The present method can be conducted for or within any suitable time. For example, the present method can be conducted for or within about an hour, e.g., conducted for or within about 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less than 1 minute.

In still another aspect, the present disclosure provides a system for assessing recombinant protein expression or reporter gene expression in a sample, which system comprises: a) a test device described above; and b) a reader that comprises a light source and a photodetector to detect a detectable signal. The present system can further comprise a barcode detector and/or a RFID detector. For example, the reader in the present system can further comprise a barcode detector and/or a RFID detector.

The principles of the present test devices, kits, systems and methods can be applied, or can be adapted to apply, to the lateral flow test devices and assays known in the art. For example, the principles of the present test devices, kits, systems and methods can be applied, or can be adapted to apply, to the lateral flow test devices and assays disclosed and/or claimed in the following patents and applications: U.S. Pat. Nos. 5,073,484, 5,654,162, 6,020,147, 4,695,554, 4,703,017, 4,743,560, 5,591,645, RE 38,430 E, 5,602,040, 5,633,871, 5,656,503, 6,187,598, 6,228,660, 6,818,455, 7,109,042, 6,352,862, 7,238,537, 7,384,796, 7,407,813, 5,714,389, 5,989,921, 6,485,982, 5,120,643, 5,578,577, 6,534,320, 4,956,302, RE 39,664 E, 5,252,496, 5,559,041, 5,728,587, 6,027,943, 6,506,612, 6,541,277, 6,737,277, 7,175,992 B2, 7,691,595 B2, 6,770,487 B2, 7,247,500 B2, 7,662,643 B2, 5,712,170, 5,965,458, 7,371,582 B2, 7,476,549 B2, 7,633,620 B2, 7,815,853 B2, 6,267,722 B1, 6,394,952 B1, 6,867,051 B1, 6,936,476 B1, 7,270,970 B2, 7,239,394 B2, 7,315,378 B2, 7,317,532 B2, 7,616,315 B2, 7,521,259 B2, 7,521,260 B2, US 2005/0221504 A1, US 2005/0221505 A1, US 2006/0240541 A1, US 2007/0143035 A1, US 2007/0185679 A1, US 2008/0028261 A1, US 2009/0180925 A1, US 2009/0180926 A1, US 2009/0180927 A1, US 2009/0180928 A1, US 2009/0180929 A1, US 2009/0214383 A1, US 2009/0269858A1, U.S. Pat. No. 6,777,198, US 2009/0311724 A1, US 2009/0117006 A1, U.S. Pat. Nos. 7,256,053, 6,916,666, 6,812,038, 5,710,005, 6,140,134, US 2010/0143941 A1, U.S. Pat. Nos. 6,140,048, 6,756,202, 7,205,553, 7,679,745, US 2010/0165338 A1, US 2010/0015611 A1, U.S. Pat. Nos. 5,422,726, 5,596,414, 7,178,416, 7,784,678 B2, US 2010/094564 A1, US 2010/0173423 A1, US 2009/0157023 A1, U.S. Pat. Nos. 7,785,899, 7,763,454 B2, US 2010/0239460 A1, US 2010/0240149 A1, U.S. Pat. Nos. 7,796,266 B2, 7,815,854 B2, US 2005/0244953 A1, US 2007/0121113 A1, US 2003/0119202 A1, US 2010/0311181 A1, U.S. Pat. Nos. 6,707,554 B1, 6,194,222 B1, 7,713,703, EP 0,149,168 A1, EP 0,323,605 A1, EP 0,250,137 A2, GB 1,526,708 and WO99/40438.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of a test strip based on lateral flow immunoassay.

FIG. 2 illustrates possible outcome of exemplary test results. C: control line. T: test line. When both C and T lines appear, the test is positive, there are expressed protein tag in the sample. When only C line appears, there is no protein expressed. If the C line do not appear, there might have been something wrong with the test and is inconclusive.

FIG. 3 illustrates possible outcome of the test results of a competition LFIA. C: control line. T: test line. When both C and T lines appear, the test is negative, there are no detectable amount of protein expressed in the sample. When only C line appears, there is protein expressed in the sample. If the C line does not appear, there might have been something wrong with the test and is inconclusive.

FIG. 4 illustrates a map of an exemplary vector used for transfection. 173H6VHH was cloned upstream of human IgG1 Fc in pcDNA 3.1 vector.

FIG. 5 illustrates exemplary test results. Human IgG was diluted from 3 ng/ml to 100 ug/ml in PBS and applied on test strips. Strip #1 to 8: 0, 3 ng/ml, 6 ng/ml, 12.5 ng/ml, 25 ng/ml, 50 ng/ml, 100 ng/ml, 200 ng/ml.

FIG. 6 illustrates exemplary test results. Cross reaction with mouse IgG and rabbit IgG was tested with the human Fc test strips. Strip 1, mouse IgG; strip 2, Rabbit IgG; Strip 3, Human IgG. 100 ng/ml of each antibody was tested with the human Fc test strips.

FIG. 7 illustrates detection of human Fc with the test strips from culture media after transfection of CHO-S cells. Time after transfection for each test strip: 1. 14 hours; 2. 24 hours; 3. three days; 4. 5 days; 5. 7 days; 6. 7 days; 7. 8 days.

FIG. 8 illustrates test strips detecting human Fc from G418 selected CHO-S cells. Time after G418 addition: 1. 2 days; 2. 4 days; 3. 5 days.

FIG. 9 illustrates competition LFIA for detection of His-tagged protein. C: control line, T1: test line 1, highest concentration; T2: test line 2, medium concentration; T3: lowest concentration. Strip 1 to 6 were tested with different concentrations of MBP-His: 1, blank; 2, 0.05 ng/ul; 3, 0.1 ng/ul; 4, 0.2 ng/ul, 5, 0.5 ng/ul, 6, 1 ng/ul.

FIG. 10 illustrates colloidal gold labeled human Fc Test strips scanned with a Qiagen ESEquant reader after reaction. 5 strips were reacted with human IgG at different concentrations (0, 3.125 ng/ml, 12.5 ng/ml, 50 ng/ml, and 200 ng/ml). On each strip, the left most peak was density of the control line, and the right peak was the density of the test line.

### DETAILED DESCRIPTION

A detailed description of one or more embodiments of the claimed subject matter is provided below along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications, including patent documents, scientific articles and databases, referred to in this application are incorporated by reference in their entireties for all purposes to the same extent as if each individual publication were individually incorporated by reference. If a definition set forth herein is contrary to or otherwise inconsistent with a definition set forth in the patents, patent applications, published applications or other publications that are herein incorporated by reference, the definition set forth herein prevails over the definition that is incorporated herein by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y.; D. Weir & C. Blackwell, eds., Handbook of Experimental Immunology (1996), Wiley-Blackwell; Cellular and Molecular Immunology (A. Abbas et al., W.B. Saunders Co. 1991, 1994); Current Protocols in Immunology (J. Coligan et al. eds. 1991), all of which are herein incorporated in their entireties by reference for all purposes.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

I. Definitions

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or VL amino acid sequence, it is understood that such a CDR has a sequence of the corresponding CDR (e.g., CDR-H3) within the variable region, as defined by any of the aforementioned schemes.

Likewise, unless otherwise specified, a FR or individual specified FR(s) (e.g., FR-H1, FR-H2), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, or Contact method.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. See, e.g., Kindt et al., Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. In particular embodiments, the antibodies are single-chain antibody fragments comprising a variable heavy chain region and/or a variable light chain region, such as scFvs.

Single-domain antibodies are antibody fragments comprising all or a portion of the heavy chain variable domain or all or a portion of the light chain variable domain of an antibody. In certain embodiments, a single-domain antibody is a camelid single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody.

A “humanized” antibody is an antibody in which all or substantially all CDR amino acid residues are derived from non-human CDRs and all or substantially all FR amino acid residues are derived from human FRs. The term “chimeric” antibody refers to an antibody in which a portion of the heavy and/or light chain is derived from a particular source or species, while the remainder of the heavy and/or light chain is derived from a different source or species.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues, and are not limited to a minimum length. Polypeptides, including the provided antibodies and antibody chains and other peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “specific binding” refers to the specificity of a binder, e.g., an antibody, such that it preferentially binds to a target, such as a polypeptide antigen. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments that are specific for or bind specifically to a target bind to the target with higher affinity than binding to other non-target substances. Also preferably, binders, antibodies or antibody fragments that are specific for or bind specifically to a target avoid binding to a significant percentage of non-target substances, e.g., non-target substances present in a testing sample. In some embodiments, binders, antibodies or antibody fragments of the present disclosure avoid binding greater than about 90% of non-target substances, although higher percentages are clearly contemplated and preferred. For example, binders, antibodies or antibody fragments of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments of the present disclosure avoid binding greater than about 10%, 20%, 30%, 40%, 50%, 60%, or 70%, or greater than about 75%, or greater than about 80%, or greater than about 85% of non-target substances.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

II. Exemplary Embodiments

In some embodiments, the present disclosure relates to the use of lateral flow immunoassay (LFIA) device to quickly detect the expression of recombinant proteins. In a preferred embodiment of the present disclosure, the LFIA device (test strip) comprises or consists of four elements (sample pad, conjugate pad, nitrocellulose membrane, absorbent pad) mounted consecutively on a solid backing. Colloidal gold nanoparticles conjugated to antibodies specific to protein or epitope tags are deposited onto the conjugate pad, which forms an immuno-complex with recombinant proteins expressing protein or epitope tags in the sample. A second antibody specific to protein or epitope tags is immobilized onto the nitrocellulose membrane. The second antibody captures the first immuno-complex. Accumulation of chromatic colloidal gold nanoparticles of the immuno-complex allows detection of recombinant protein expression.

In some embodiments, the present disclosure uses LFIA to quickly detect the expression of recombinant proteins through protein or epitope tags. Protein tags and epitope tags are often used as part of the recombinant protein for various purposes, such as affinity purification, immunoprecipitation, tracing of expression. Many protein tags also improve the solubility of expressed protein and increase the production yield. Protein and epitope tags are commonly used in all in vivo recombinant expression systems, such as bacterial, yeast, insect, mammalian and plant, etc. In vitro translation systems also use these tags. Commonly used protein tags includes: Fc domain of IgG (human, rabbit, mouse), chitin binding protein (CBP), maltose binding protein (MBP), glutathione-S-transferase (GST), etc. Commonly used epitope tags includes: poly Histidine, FLAG, HA, c-myc, V5, Avi Tag, Strep II tag, etc. [1-6].

In some embodiments, the objective of the present disclosure is to design a simple, sensitive, cost-effective device for the rapid detection of recombinant protein expression. It is possible to achieve these objectives by use of lateral flow immunoassay (LFIA) device.

In some embodiments, the present disclosure is directed to the application of exemplary lateral flow immunoassay device to detect recombinant protein expression. LFIA devices detecting protein/epitope tags are able to rapidly determine the recombinant protein expression levels in any expression systems if the protein is expressed with the tag. Such devices can be used to quickly determine protein expression directly from sample lysates. The LFIA devices could be made to be qualitative, semi-quantitative, or quantitative.

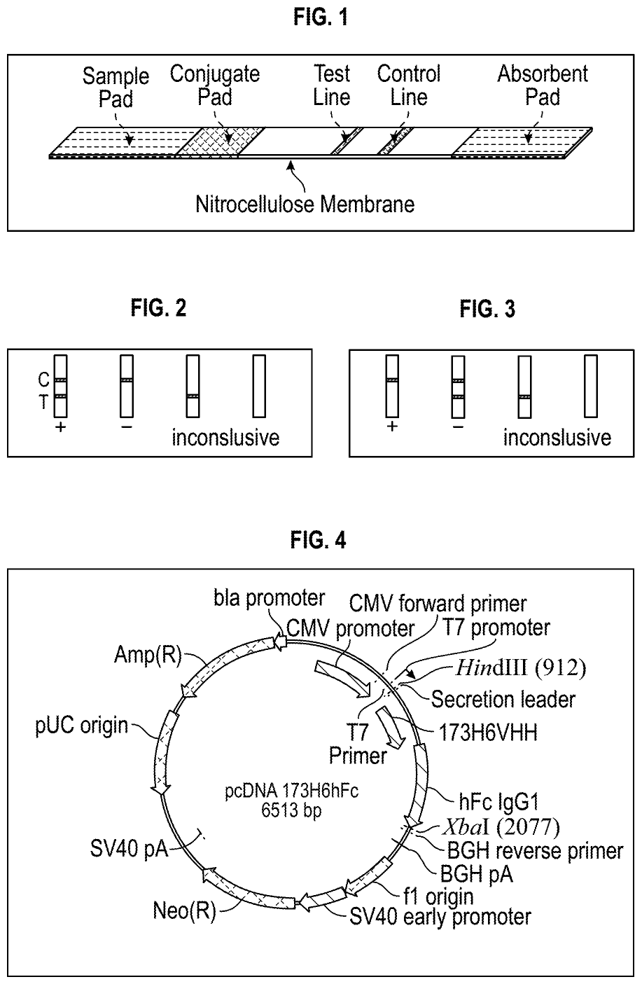

In some embodiments, the exemplary lateral flow immunoassay device comprises or consists of four elements: sample pad, conjugate pad, nitrocellulose membrane, absorbent pad. It is essentially a series of four capillary beds capable of transporting fluid samples spontaneously through capillary actions. The first element is sample pad, a porous membrane where samples are applied. In the second element (conjugate pad), colloidal gold nanoparticles conjugated to antibodies specific to protein or epitopes tags are deposited (FIG. 1). When conjugate pad receives samples from the sample pad, the antibody conjugated with colloidal gold nanoparticles rehydrates and binds to specific protein or epitope tags, forming the first immuno-complex. The sample along with the first immuno-complex continues to migrate towards the nitrocellulose membrane (the third element) where a second antibody specific to protein or epitope tags is deposited and immobilized within a narrow band on the nitrocellulose membrane forming test lines. As more immuno-complexes passing through and being captured by the second antibody, the color of the test lines intensified due to accumulation of chromatic nanogold particles of the immuno-complex (FIG. 2). This allows rapid detection of recombinant protein expression. The fourth element (absorbent pad) acts as a waste reservoir and directs the capillary flow.

In some embodiments, potential applications of our LFIA recombinant protein detection device includes: 1) rapid high-throughput screening (HTS) large number of samples (such as different clones) to determine the best candidate with higher production yield; 2) protein expression time course determination; 3) protein expression induction condition optimization; and 4) Rapidly determine the fractions with intended protein during protein purification/chromatography.

In a preferred embodiment, the antibody label may be any chromatic materials, e.g., colloidal gold nanoparticles, quantum dots, a colored enzyme, or a fluorescent particle.

In a preferred embodiment, the first antibody may be specific to a first epitope or a first tag of the recombinant protein; and the second antibody may be specific to the first or the second epitope or tag of the recombinant protein. Either of the antibodies could be combinations of polyclonal or monoclonal antibodies.

In some embodiments, the lateral flow could also be a competition type of assay, where the epitope tag specific antibody is conjugated to the colloidal gold nanoparticle, and the test line on the solid phase is a competitor protein with the same detection epitope. When there is no intended protein/tag present in the sample, the antibody-gold nanoparticle complex will migrate to the test line on the solid phase and bind on the test line, where a colored line will appear to indicate negative results. When there is intended protein presence in the sample, e.g., the epitope tag, the sample will bind the antibody on the gold nanoparticle and migrate to the test line. The epitope tag on the test line will compete the gold nanoparticle therefore no test line will appear, indicating positive results. FIG. 3 illustrates the typical results from a competition LFIA. Multiple test lines with different concentrations of competitors could be printed to facilitate signal detection (semi-quantitative).

Further Exemplary embodiments are described below in detail with reference to the accompanying drawings. FIG. 1 is a schematic diagram of the LFIA device. The LFIA device (test strip) comprises or consist of four elements: sample pad, conjugate pad, nitrocellulose membrane, absorbent pad. These four elements are mounted consecutively on a solid backing. The first element of the LFIA device is sample pad. It is located on one end of the LFIA test strip. It is a porous membrane where samples are applied. Once the sample is dispensed on the sample pad, the sample migrate toward the other end of the LFIA test strip due to capillary action.

The second element is a conjugate pad. The presence of recombinant proteins with protein or epitope tags is detected by antibodies specific to the protein or epitope tag. Colloidal gold nanoparticles conjugated to antibodies serve as chromatic signals for detection. Monodispersed colloid gold nanoparticles are attractive choice for detection due to its sensitivity and tunable vibrant color. The color of colloidal gold nanoparticle strongly depends on its environment and its physical size. In the exemplary embodiments of the present invention, colloidal gold nanoparticles conjugated to antibodies specific to protein or epitopes tags are deposited into the conjugate pad. When conjugate pad receives samples from the sample pad, the antibody conjugated with colloidal gold nanoparticles rehydrates and binds to specific protein or epitope tags, forming the first immuno-complex.

The sample along with the first immuno-complex continues to migrate towards the nitrocellulose membrane (the third element). A second antibody specific to protein or epitopes tags is deposited and immobilized within a narrow band on the nitrocellulose membrane forming test lines. Additional control line could also be added onto the nitrocellulose membrane for quality control. As more immuno-complexes passing through and being captured by the second antibody, the color of the test lines intensified due to accumulation of chromatic nanogold particles of the immuno-complex. This allows rapid detection of recombinant protein expression. The fourth element (absorbent pad) acts as a waste reservoir. It also prevents back flow of the sample as it continues to draw samples in.

FIG. 2 shows interpretation of possible test results of LFIA device. The control line indicate whether the LFIA is function properly. The test line indicates the presence or absence of the recombinant protein with protein/epitope tag. The intensity of the test line could be determined with image processing program, providing quantitative analysis of recombinant protein expression. FIG. 3 shows interpretation of possible test results of competition LFIA.

Other exemplary applications. The test strips can be used in many other applications, including but not limited to: selection of positive clones that express intended protein after transfection or transformation, determine time course of protein expression, induction condition optimization for protein expression, or determine fractions containing desired protein in fractions collected from chromatography. Quantitative analysis is also possible if a strip reader is available, such as the Qiagen ESEquant reader (FIG. 10). The example shows a quantitative plot of colloidal gold labeled test strips. Other labels such as different fluorescent dyes can also be used in quantitation.

The test strips can also be used to detect reporter gene expression when reporter specific antibodies are used in the LFIA, such as luciferase or alkaline phosphatase.

In some embodiments, the present disclosure provides a lateral flow immunoassay device for qualitative or quantitative analysis of recombinant protein expression using protein or epitope tags. The described disclosure is applicable to detect protein samples from all recombinant protein expression systems including: bacterial, yeast, insect, mammalian and plant, etc. It is also applicable to in vitro recombinant protein expression system.

### EXAMPLES

### Example 1: Rapid Detection of Human Fc Fusion Protein Expression in Chinese Hamster Ovary Cells

### Preparation of the Conjugate.

Preparation of colloidal gold nanoparticles. Colloidal gold nanoparticles were prepared by reduction of chloroauric acid, similar to the method by Turkevich (J. Turkevich, P. C. Stevenson, J. Hillier, “A study of the nucleation and growth processes in the synthesis of colloidal gold”, Discuss. Faraday. Soc. 1951, 11, 55-75) and Frens (G. Frens, “Particle size and sol stability in metal colloids”, Colloid & Polymer Science 1972, 250, 736-741) [7].

Preparation of the antibody-colloidal gold nanoparticles conjugate. Antibody-colloidal nanogold conjugate was prepared by a method similar to those by Bailes (Bailes, J., et al., Effect of gold nanoparticle conjugation on the activity and stability of functional proteins. Methods Mol Biol, 2012. 906: p. 89-99; and Bailes, J., et al., Gold nanoparticle antibody conjugates for use in competitive lateral flow assays. Methods Mol Biol, 2012. 906: p. 45-55) [8, 9].

### Assembly of LFIA Test Strip

LFIA test strips were assembled by mounting the four LFIA elements (sample pad, conjugate pad, nitrocellulose membrane, absorbent pad) consecutively on a solid backing. The goat anti-human IgG was conjugated to the colloidal gold and printed on the conjugate pad. The test line was printed with goat anti-human IgG. The control line was printed with Donkey anti-goat IgG.

### Test Samples

In this example, the coding region of a single domain antibody from llama was cloned upstream of the human IgG1 Fc domain in the pcDNA3.1 vector (FIG. 4). Plasmid DNA was purified and transfected to actively growing Freestyle CHO-S cells (Thermofisher cat #R80007) in a 125 ml shaker flask. Cell culture media were collected at different time points after transfection. The presence of human Fc in each media fraction was detected with test strips specific for human Fc.

### Assay Results

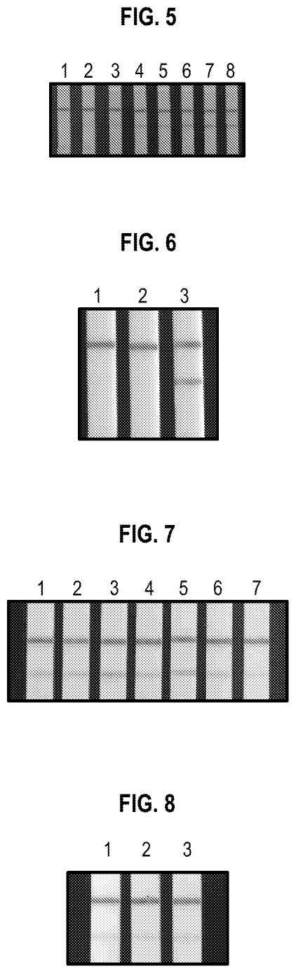

Sensitivity. Human IgG was used as a positive control, the culture media was used as a negative control. Human IgG was diluted to various concentrations from 3 ng/ml to 1 ug/ml. Each sample was tested with the test strip (FIG. 5). The detection limit is well below 3 ng/ml, much lower than normally productive expression system, which would yield specific proteins in ug/ml range or higher.

Specificity. Cross reaction with mouse IgG and rabbit IgG was tested with the human Fc test strips (FIG. 6). There are no cross reaction with mouse or rabbit IgG at 100 ng/ml concentration.

Culture media samples collected at different time points from transfected CHO-S cells were tested with test strips. The expression of the protein (presence of Fc) is detectable as early as 14 hours post transfection (FIG. 7). Transfected CHO-S cells were also cultured in the presence of 600 ug/ml G418 to select stable drug resistant cells, with starting 5×10e5/ml CHO-S cells two days after transfection. The culture media were changed every two days in the G418 selected pools. The presence of human Fc was detected with test strips (FIG. 8). On the 8^{th} day post transfection, the transiently transfected cells (non-drug selected) has reach 8×10e6/ml density. The G418 selected cells reached 1.2×10e6/ml. However, the amount of expressed protein in the drug selected cells reached to similar level as that in the transient transfected cells with lower cell density, indicating an enrichment of cell population with expression of human Fc. This test strips can be used to monitor the stable cell selection for recombinant protein expression.

### Example 2: Rapid Detection of Human Fc Fusion Protein Expression in Chinese Hamster Ovary Cells

### Preparation of the Conjugate.

Preparation of colloidal gold nanoparticles. Similar to example 1, colloidal gold nanoparticles were prepared by reduction of chloroauric acid, similar to the method by Turkevich and Frens [7].

Preparation of the antibody-colloidal gold nanoparticles conjugate. Similar to example 1, antibody-colloidal nanogold conjugate was prepared by a method similar to those by Bailes [8, 9].

### Assembly of LFIA Test Strip

LFIA test strips were assembled by mounting the four LFIA elements (sample pad, conjugate pad, nitrocellulose membrane, absorbent pad) consecutively on a solid backing. A monoclonal mouse anti-His tag antibody was conjugated to the colloidal gold and printed on the conjugate pad. Three test lines were printed with poly-His peptide conjugated BSA at different concentrations. The control line was printed with goat anti-mouse antibody.

### Test Samples

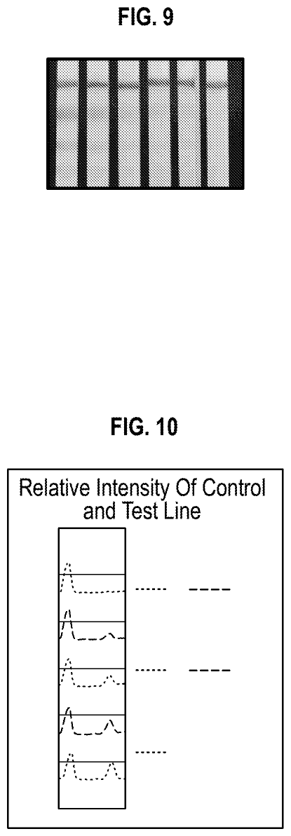

In this example, a maltose binding protein (MBP) with poly-His tag at the C-terminus of the protein was expressed in a pET22b vector. Purified His-tagged MBP was diluted to different concentrations and tested with the His tag test strips (FIG. 9). At 0.05 ng/ul, the T3 line started to disappear. As the His tagged protein increase, all test lines gradually fade, and at 10 ng/ul, all test lines disappeared.

References cited:
1. Einhauer, A. and A. Jungbauer, The FLAG peptide, a versatile fusion tag for the purification of recombinant proteins. J Biochem Biophys Methods, 2001. 49(1-3): p. 455-65.
2. Keefe, A. D., et al., One-step purification of recombinant proteins using a nanomolar-affinity streptavidin-binding peptide, the SBP-Tag. Protein Expr Purif, 2001. 23(3): p. 440-6.
3. Prakriya, M., et al., Orai1 is an essential pore subunit of the CRAG channel. Nature, 2006. 443(7108): p. 230-3.
4. McNutt, M. C., T. A. Lagace, and J. D. Horton, Catalytic activity is not required for secreted PCSK9 to reduce low density lipoprotein receptors in HepG2 cells. J Biol Chem, 2007. 282(29): p. 20799-803.
5. Beckett, D., E. Kovaleva, and P. J. Schatz, A minimal peptide substrate in biotin holoenzyme synthetase-catalyzed biotinylation. Protein Sci, 1999. 8(4): p. 921-9.
6. Brizzard, B., Epitope tagging. Biotechniques, 2008. 44(5): p. 693-5.
7. Kimling, J., et al., Turkevich method for gold nanoparticle synthesis revisited. J Phys Chem B, 2006. 110(32): p. 15700-7.
8. Bailes, J., et al., Effect of gold nanoparticle conjugation on the activity and stability of functional proteins. Methods Mol Biol, 2012. 906: p. 89-99.
9. Bailes, J., et al., Gold nanoparticle antibody conjugates for use in competitive lateral flow assays. Methods Mol Biol, 2012. 906: p. 45-55.

1. A lateral flow test device for assessing recombinant protein (polypeptide) expression or reporter gene expression in a sample, which device comprises a porous matrix that comprises a test location on said porous matrix, said test location comprising a test reagent that binds to an analyte or another binding reagent that binds to said analyte, or is an analyte or an analyte analog that competes with an analyte in said sample for binding to a binding reagent for said analyte,
wherein said analyte is a recombinant protein (polypeptide) expression product or a reporter gene expression product, and
wherein a liquid sample flows laterally along said test device and passes said test location to form a detectable signal to determine the presence, absence and/or amount of said recombinant protein (polypeptide) expression product or said reporter gene expression product in said sample.

2. (canceled)

3. The test device of claim 1, wherein the test reagent binds to the analyte.

4-6. (canceled)

7. The test device of claim 1, wherein the test reagent is a peptide or a protein.

8-14. (canceled)

15. The test device of claim 1, wherein a portion of the matrix, upstream from the test location, comprises a dried, labeled reagent, the labeled reagent being capable of being moved by a liquid sample and/or a further liquid to the test location and/or the control location to generate a detectable signal.

16-31. (canceled)

32. The test device of claim 15, wherein the test reagent and the labeled reagent bind to the analyte, and at least one of the test reagent and the labeled reagent specifically binds to the analyte.

33-37. (canceled)

38. The test device of claim 1, which is configured for assessing recombinant protein expression in a sample.

39-42. (canceled)

43. The test device of claim 38, wherein the test reagent is the recombinant protein expression product, or a portion thereof, that competes with the recombinant protein expression product in the sample for binding to the labeled reagent that binds to, or specifically binds to, the recombinant protein expression product.

44-46. (canceled)

47. The test device of claim 1, which is configured for assessing reporter gene expression in a sample.

48-51. (canceled)

52. The test device of claim 47, wherein the test reagent is the reporter gene expression product, or a portion thereof, that competes with the reporter gene expression product in the sample for binding to the labeled reagent that binds to, or specifically binds to, the reporter gene expression product.

53-55. (canceled)

56. The test device of claim 1, which is configured for:
1) assessing multiple recombinant protein expression products or multiple reporter gene expression products in a sample;
2) determining a candidate for recombinant protein expression with intended production yield;
3) assessing recombinant protein expression or reporter gene expression time course;
4) optimizing induction condition for recombinant protein expression or reporter gene expression; and/or
5) determining the fraction(s) with intended recombinant protein during protein purification, e.g., chromatography.

57. A method for assessing recombinant protein expression or reporter gene expression in a sample, which method comprises:
a) contacting a liquid sample with the test device of claim 1, wherein the liquid sample is applied to a site of the test device upstream of the test location;
b) transporting the analyte, if present in the liquid sample, and a labeled reagent to the test location; and
c) assessing a detectable signal at the test location to determine the presence, absence and/or amount of said recombinant protein expression product or said reporter gene expression product in said sample.

58. The method of claim 57, wherein the liquid sample and the labeled reagent are premixed to form a mixture and the mixture is applied to the test device.

59-61. (canceled)

62. The method of claim 57, wherein the test device comprises a dried labeled reagent before use and the dried labeled reagent is solubilized or resuspended, and transported to the test location by the liquid sample.

63-66. (canceled)

67. The method of claim 57, wherein the liquid sample comprises a cell lysate, a cell culture medium, an in vitro transcription product, an in vitro translation product, a polypeptide purification fraction, and/or a sample isolated or derived from a subject.

68. The method of claim 57, wherein the detectable signal is assessed by a reader.

69-71. (canceled)

72. The method of claim 57, which is conducted for assessing a recombinant protein expression in a sample.

73. (canceled)

74. (canceled)

75. The method of claim 57, which is conducted for assessing a reporter gene expression in a sample.

76. (canceled)

77. (canceled)

78. The method of claim 57, which is conducted for:
1) assessing multiple recombinant protein expression products or multiple reporter gene expression products in a sample;
2) determining a candidate for recombinant protein expression with intended production yield;
3) assessing recombinant protein expression or reporter gene expression time course;
4) optimizing induction condition for recombinant protein expression or reporter gene expression; and/or
5) determining the fraction(s) with intended recombinant protein during protein purification, e.g., chromatography.

79. The method of claim 57, which is conducted for or within about an hour.

80. A system for assessing recombinant protein expression or reporter gene expression in a sample, which system comprises:
a) a test device of claim 1; and
b) a reader that comprises a light source and a photodetector to detect a detectable signal.

81. (canceled)
